(19) United States
(12) Patent Application Publication
(10) Pub. No.: US 2026/0234525 A1
(43) Pub. Date: Aug. 13, 2026

(54) Methods for Purging Bubbles from Cultureware Microfeatures

(71) Applicant: XDemics Corporation, Novato, CA (US)

(72) Inventors: Colin A. Cook, Oakland, CA (US); Nicholas E. Scianmarello, San Francisco, CA (US); Austin R. Santiago, Novato, CA (US); Christopher D. Thanos, Tiburon, CA (US)

(73) Assignee: XDemics Corporation, Novato, CA (US)

(21) Appl. No.: 19/536,256

(22) Filed: Feb. 11, 2026

Related U.S. Application Data
(60) Provisional application No. 63/757,456, filed on Feb. 12, 2025.
(60) Provisional application No. 63/770,927, filed on Mar. 12, 2025.
(60) Provisional application No. 63/874,207, filed on Sep. 2, 2025.

(51) Int. Cl. H10D 30/01; H10D 84/01; H10D 84/83; H10D 84/85

(52) U.S. Cl. CPC C12M 23/34; C12M 23/02; C12M 23/12; C12M 23/24; C12M 33/08; B01L 2300/08; B01L 2300/0851; B01L 2400/086

(57) ABSTRACT

Micro fences that extend laterally between slightly larger fins on the bottom of gas permeable, hydrophobic cultureware can help purge air that may otherwise get trapped between the fins. The low-rise fences may be placed within longitudinal gaps between fins or between the fins themselves. The fins may have rounded or slanted tops, longitudinal slopes, chamfered ends, and/or other surface features to help free bubbles upward or into a peripheral groove that surrounds the bottom of the cultureware. The fins themselves may vary in width along their lengths to form pinch points and create wider regions in order to allow bubbles to find each other and merge. The bottoms of the grooves between the fins may undulate along their lengths to form peaks and valleys where bubbles may gather and release.

### CROSS-REFERENCES TO RELATED APPLICATIONS

This application claims the benefit of U.S. Provisional Patent Application No. 63/757,456, filed 12 Feb. 2025, U.S. Provisional Patent Application No. 63/770,927, filed 12 Mar. 2025, and U.S. Provisional Patent Application No. 63/874,207, filed 2 Sep. 2025, the contents of which are hereby incorporated by reference in their entireties for all purposes.

### STATEMENT AS TO RIGHTS TO INVENTIONS MADE UNDER FEDERALLY SPONSORED RESEARCH AND DEVELOPMENT

Not applicable.

### BACKGROUND

### 1. Field of the Invention

The present application generally relates to containers or dishes for laboratory use in retaining material to be analyzed with fluid transport features. Specifically, the application is related to petri dishes, single-and multi-well plates, and other cultureware with miniature elongated bottom fin features defining grooves interrupted by low-rise fences, peaks within the groove floors, horizontally necked off areas, or other narrowing features that facilitate bubble removal.

### 2. Description of the Related Art

Gas permeable cultureware and bioproduction devices can have surface features to improve gas exchange, increase surface area for adherent cells, create microwells for spheroid generation, and provide niches to protect cells from shear forces. When fabricated from silicone or other hydrophobic materials, surface features can create pockets that are challenging to wet and result in entrapped air and bubbles.

U.S. Pat. No. 11,981,885 discloses cultureware with a gas-permeable membrane on the bottom that has elongated surface features that physically protect cells and channel oxygen to them. Such devices work best when air bubbles are removed in a timely manner, ideally before cells settle into the niches, and preferably without user involvement.

There is a need in the art for methods of purging entrapped bubbles from surface features in cultureware.

### BRIEF SUMMARY

Generally, laboratory cultureware is described that includes elongated, gas-permeable fin features along the bottom of its liquid holding area that define grooves in between then, along with narrowing features within the grooves where air bubbles can merge and be dispelled. Discrete, low-rise barriers can extend horizontally between the fins, or the groove floor can gently slope up into peaks. The barriers or peaks can exist in the gaps between fins or deep within the grooves. Alternatively or in addition, the fins themselves can widen in some areas, correlating with neighbors, providing a choke point within the grooves. Air bubbles can be expelled upward or into a peripheral groove.

The tops of the fins can slope up from one end to another, such that an air bubble in a groove will work its way up to the highest point of the adjacent fin. The top of a fin can be rounded or slanted, or the entire fin's height tapered, so that a bubble can work its way out of the groove.

Some embodiments of the present invention are related to a cultureware vessel apparatus including a basin bed, an array of fins projecting upward from the basin bed, the array having rows and columns of the fins, each row of the rows formed from longitudinally aligned fins of the array and gaps therebetween, each column of the columns formed from fins in separate rows and grooves therebetween, a low-rise fence projecting upward from the basin bed to a height below a top of the fins, the low-rise fence extending laterally between two of the rows, and a sidewall surrounding the basin bed.

The low-rise fence can extend between two of the gaps. The low-rise fence can extend between a pair of the fins, such as at a midpoint. The low-rise fence can be a first low-rise fence, and there can exist a second low-rise fence extending between the pair of fins. A height of the first and second low-rise fences can be at least tan(5°) multiplied by a distance between the first and second row-rise fences.

Each fin of the fins can have a top that slopes along a longitudinal length of the fin. A column of the columns can have tops of fins that slope in a same direction. Alternating columns of the fins can have opposite slopes such that a high side of a first column of fins is adjacent a high side of an adjacent column of fins, and a low side of the first column of fins is adjacent a low side of an opposite adjacent column of fins.

A width of each fin can vary along its length, a maximum width between neighboring fins occurring at a same longitudinal location to form a pinch point in the groove therebetween. The pinch points in multiple of the grooves can align laterally. A bottom of each groove of the grooves can vary in height to form a peak along the groove. The peaks along the grooves can align laterally. A height of the low-rise fence can be less-than-or-equal-to half of a height of the fins, such as one fifth. At least one of the fins can have a height between 400 μm and 1000 μm, a width between 300 μm and 500 μm, and a pitch between 200 μm and 500 μm. At least one of the fins can have a height between 700 μm and 1000 μm. The average diameter of a biological cell of interest is about 20 μm.

Longitudinal ends of the fins can be rounded, tapered, chamfered, or other geometries. The basin bed, the fins, and the fence can be formed from a gas-permeable resilient polymer. A width of each fin can taper from a bottom to a top of the fin. A peripheral groove adjacent the sidewall can surround all of the fins. The peripheral groove can be deeper than the grooves between the rows.

Some embodiments are related to a method of manufacturing a cultureware vessel, the method including forming a basin bed, forming an array of fins projecting upward from the basin bed, the array having rows and columns of the fins, each row of the rows formed from longitudinally aligned fins of the array and gaps therebetween, each column of the columns formed from fins in separate rows and grooves therebetween, forming a low-rise fence projecting upward from the basin bed to a height below a top of the fins, the low-rise fence extending laterally between two of the rows, and forming a sidewall surrounding the basin bed.

Some embodiments are related to a cultureware vessel apparatus including a basin bed, a plurality of fins projecting upward from the basin bed and substantially parallel with each other, the fins defining grooves therebetween, and a sidewall surrounding the basin bed, wherein a width of the fins varies along its length, a maximum width between neighboring fins occurring at a same longitudinal location to form a pinch point in the groove between the neighboring fins.

Pinch points in multiple of the grooves can align laterally.

Some embodiments are related to a method of manufacturing a cultureware vessel, the method including forming a basin bed, forming a plurality of fins projecting upward from the basin bed and substantially parallel with each other, the fins defining grooves therebetween, and forming a sidewall surrounding the basin bed, wherein a width of the fins varies, a maximum width between neighboring fins occurring at a same longitudinal location to form a pinch point in the groove between the neighboring fins.

Some embodiments are related to a cultureware vessel apparatus including a basin bed, a plurality of fins projecting upward from the basin bed and substantially parallel with each other, the fins defining grooves therebetween, and a sidewall surrounding the basin bed, wherein a bottom of each groove of the grooves varies in height to form a peak along the groove.

Peaks along the grooves can align laterally.

Some embodiments are related to a method of manufacturing a cultureware vessel, the method including forming a basin bed, forming a plurality of fins projecting upward from the basin bed and substantially parallel with each other, the fins defining grooves therebetween, and forming a sidewall surrounding the basin bed, wherein a bottom of each groove of the grooves varies in height to form a peak along the groove.

The molding operations can be performed all at one time or at separate times and in various orders.

### BRIEF DESCRIPTION OF THE DRAWINGS

FIG. 1A is a top-side perspective view of a one-well cultureware with miniature fins and grooves in accordance with an embodiment.

FIG. 1B is a close up top-side perspective view of the fins and grooves of FIG. 1A.

FIG. 2 is a top-side perspective view of fins and grooves with rounded low-rise fences in accordance with an embodiment.

FIG. 3A is a perspective view of low-rise fences between fins in accordance with an embodiment.

FIG. 3B is a top view of the low-rise fences between fins of FIG. 3A.

FIG. 3C is a cross-section view of the low-rise fences between fins of FIG. 3A. It is a “view” in that a fin in the background is visible, for scale.

FIG. 4 is a cross-section of low-rise fences being tilted, in accordance with an embodiment.

FIG. 5 is a cross-section of tapered fins in accordance with an embodiment.

FIG. 6 is a top-side perspective view of longitudinally sloping fins in accordance with an embodiment.

FIG. 7A is a perspective view of a petri dish with fins and grooves in accordance with an embodiment.

FIG. 7B is a top view of the petri dish of FIG. 7A.

FIG. 7C is a cross-section perspective view of the petri dish of FIG. 7A.

FIG. 8 is a cross-section perspective view of diamond-shaped grooves in accordance with an embodiment.

FIG. 9 is a cross-section perspective view of a groove with an undulating floor in accordance with an embodiment.

FIG. 10 is a flowchart illustrating a process in accordance with an embodiment.

FIG. 11 is a flowchart illustrating a process in accordance with an embodiment.

FIG. 12 is a flowchart illustrating a process in accordance with an embodiment.

### DETAILED DESCRIPTION

Cultureware with tiny elongated protrusions projecting upward from the basin bed and sized and otherwise configured to protect biological cells in grooves between the protrusions—can trap air when wetted. The air gets trapped among all of the surface area in the grooves. The trapped air is a symptom that it is sometimes more energetically favorable for air bubbles to adhere to surfaces than it is for them to break free and dissipate. This can occur in cultureware spanning all sorts of sizes, such as 2, 3, 4, 6, 12, 24, 96, 384, or 1536-well plates, petri dishes, and large T-flasks.

Air bubbles are largely unwanted for culturing cells and other uses of such cultureware. A consistently wetted surface is valued. Therefore, a variety of techniques have been tried and tested to purge air bubbles and evenly wet the surfaces. Centrifugation with or without plate wedges or curved bottoms have been tried. Surface coatings, such as polyethylene glycol (PEG)-Siloxane can also work to some extent, as well as media modifiers and wetting fluids. Sacrificial fillers, such as dissolvable polyvinyl alcohol (PVA) material, can work between the fins. Surface modifications to increase their hydrophilicity can be achieved with plasma and other treatments. Impregnation of the bulk material with amphiphilic or hydrophilic wetting molecules can be effective. Vacuum and pressure manifolds can be used, as well as stretching, compressing, and deforming of resilient cultureware, to move and disperse air bubbles. Agitation, shaking, and triturating are relatively simple methods to dislodge bubbles. Autoclaving and hotplating can also be employed. There are downsides with each of the methods above. A focus of the present disclosure is niche geometries that help to purge bubbles.

With niche geometries, the shape of surface features can be engineered to more readily wet and de-bubble. They do not require chemical additives, additional manufacturing steps, or end user intervention, to a large extent. For example, the addition of a peripheral groove can provide a pathway for air to escape around the entire device. This peripheral groove can be connected to other cavities, so they are fluidically connected. This facilitates a wetting front in transversing the surface.

Cavities within the device can similarly be fluidically connected to one another. For cavities that need to be physically separated to isolate cells, a partial height fence can be used that accomplishes cell isolation while maintaining a fluidic path to other cavities.

Bubbles will tend to migrate in the direction that minimizes their surface energy for any given volume, that is: γ=γ_{sg}A_{sg}+γ_{lg}A_{lg}, where γ is the total surface energy, γ_{sg} is the interfacial energy between the solid and gas, γ_{lg} is the interfacial energy between the gas and liquid, A_{sg} is the area of the gas bubble in contact with the solid, and A_{lg} is the area of the gas bubble in contact with the liquid. If γ_{lg}>γ_{sg}, a hydrophobic surface, then the area of the bubble in contact with the solid should increase at the expense of the solid area in contact with the liquid. A bubble would therefore migrate or deform to achieve this. If γ_{lg}<γ_{sg}, a hydrophilic surface, the inverse should occur.

For a hydrophilic gas permeable microfeature, a tapered microwell could result in a bubble climbing out of the microfeatures. Oftentimes, bubbles become entrapped in surface features due to a symmetry; while it may be energetically favorable for the bubble to be displaced, there is nothing to break the symmetry and force the bubble out one side or the other. The introduction of asymmetrical features can mitigate this problem. For example, otherwise round holes can be made in a teardrop shape. In another example, grooves, fins, or recesses can be made to have topographical undulations (e.g. sinusoidal, zig-zagged, chevron). In a further example, grooves can feature thinning gaps. Specific regions of the device can feature nucleation sites (e.g. texture, sharp features, material composition) to preferentially generate bubbles away from key geometries.

The cultureware device can be placed at an angle to provide a differential hydrostatic pressure over preferred surface features. As an example, during seeding of cells and media, the device can be held at an angle to facilitate the displacement of bubbles out of surface features; the angle can break symmetry that keeps bubbles entrapped.

An evenly wetted surface can be important for many industrially useful biological cells. For example, they can include those for the production of biological products, such as proteins and viruses, using the following cell lines: Chinese Hamster Ovary (CHO), Human Embryonic Kidney (HEK)293, Spodoptera frugiperda (SF)9, A549 adenocarcinomic human alveolar basal epithelial cells, Henrietta Lacks (HeLa), Vero African green monkey, baby hamster kidney (BHK), human cell line PER. C6, Madin-Darby Canine Kidney (MDCK), hybridomas, bacterial cells, yeast cells, plant cells, fungal cells, insect cells and immortalized cell lines.

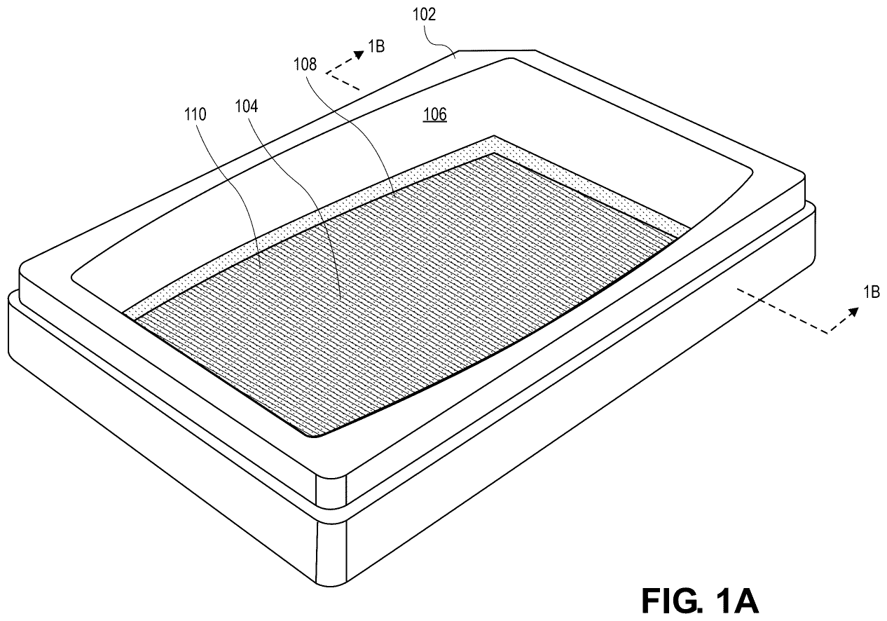

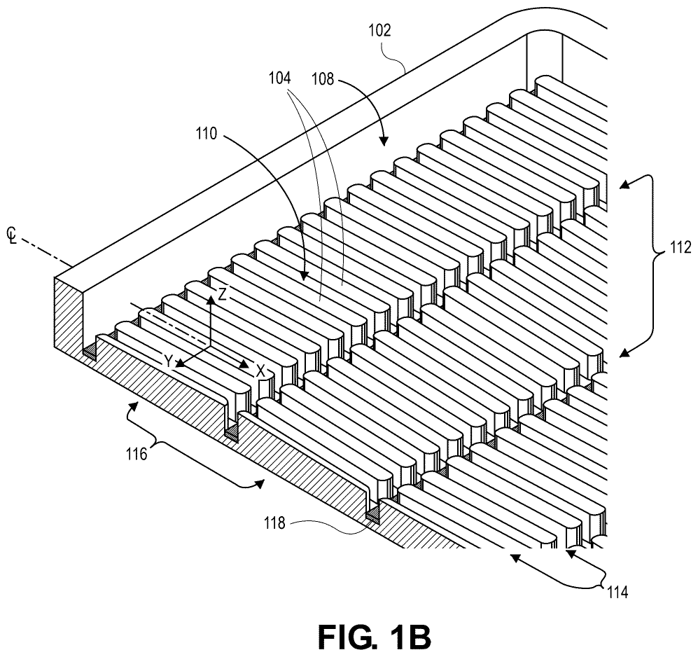

FIGS. 1A-B illustrate a one-well cultureware vessel 102. Cultureware 102 includes basin base 108 surrounded by sidewall 106 to form a vessel sufficient to hold water or other liquids.

Projecting upward from base 108 are miniature fins 104. In the exemplary embodiment, fins 104 are parallel to one another and are all at a single height. They are formed from the same material as the rest of basin base 108, a clear, gas-permeable silicone rubber. The height of fins is between 700 μm and 1000 μm.

Fin heights between 400 μm and 1000 μm, widths between 300 μm and 500 μm, and pitches between 200 μm and 500 μm tend to work well if the bottom basin and fins are made of gas-permeable silicone rubber. These dimensions allow diatomic oxygen and carbon dioxide to pass through a thin bottom and be channeled through the fins. What also works around this scale is where each one of the fins has a ratio of height to width to pitch of 4-10 to 3-5 to 2-5.

FIG. 1B shows a fin upon which three orthogonal axes are placed. An x-axis runs longitudinally along the length of the fin and parallel with an overall bottom plane of the vessel. A y-axis projects laterally and parallel with the bottom plane. A z-axis projects vertically upward, normal to the bottom plane. A centerline drawn parallel with the x-axis confirms that the fin is symmetric around the xz-plane.

Each neighboring pair of fins 104 defines a groove 110 that lies between them. In the exemplary embodiment, the grooves have flat bottoms, except for the low-rise fences described below.

Following each fin to its longitudinal (x-axis) end, a gap 112 separates it from the longitudinal end of another fin. That is, the fins in the same line share a common longitudinal axis with gaps 112 in between.

Fins 104 are arrayed in rows 114 and columns 116. Each row in rows 114 is defined by a set of fins in the same longitudinal (x-axis) line along with gaps 112. The gaps 112 between said fins are considered as part of the same row. Rows are separated by grooves 110.

Each column in columns 116 is defined by a set of fins that are in the same lateral (y-axis) line. Respective grooves 110 between said fins can be considered in the same column. Columns are separated by gaps 112.

In some embodiments, each row's fins and gaps can be offset from a neighboring row's fins and gaps. The exemplary embodiment shows the fins and gaps all aligned.

Low-rise fences 118 project upward from basin base 108. The fences are all the same height, which is below the common height of fins 104. In the exemplary embodiment, the fences are ⅕ of the height of the fins. The fences extend laterally between rows 114 by extending between two gaps 112. These can be referred to as inter-gap fences.

In some embodiments, low-rise fences can extend directly between a pair of fins such that the ends of each fence abuts against a side of a respective fin. These can be referred to as an inter-fin fences. There can be multiple such inter-fin fences in the groove between two fins, and they can be spaced quite tightly with one another. For example, they can be placed at a pitch of 0.5, 1, 1.5, 2, 2.5, 3, 3.5, 4, 4.5, 5 millimeters (mm) within the grooves. Further, embodiments can employ both inter-fin fences and inter-gap fences in the same basin bed.

There can be embodiments where there are inter-fin fences in only a subarea of the basin bed, such as the center or along one wall. Meanwhile, inter-gap fences can be spread out along the entire basin bed. The small area of the basin bed with inter-fin fences may be an area that would otherwise suffer from excess air entrapment, such as in the center of a large area.

A technical advantage of the low-rise fences is in that air bubbles tend to deform and break away from a groove when encountering the fences. The fences, along with gaps between the rows, direct bubbles along surfaces such that they can make their way to other grooves, combine with other bubbles, then rise out of the surrounding liquid.

In may embodiments, the fences do not need to be greater than half of the height of the fins for there to be a noticeable effect. Heights between ⅕ and ½ of the height of the fins have been shown to work. Lower heights, such as 40 microns (μm), can work as well because they are about twice the height of certain biological cells of interest. Higher fence heights, e.g., 400 μm, can also work effectively.

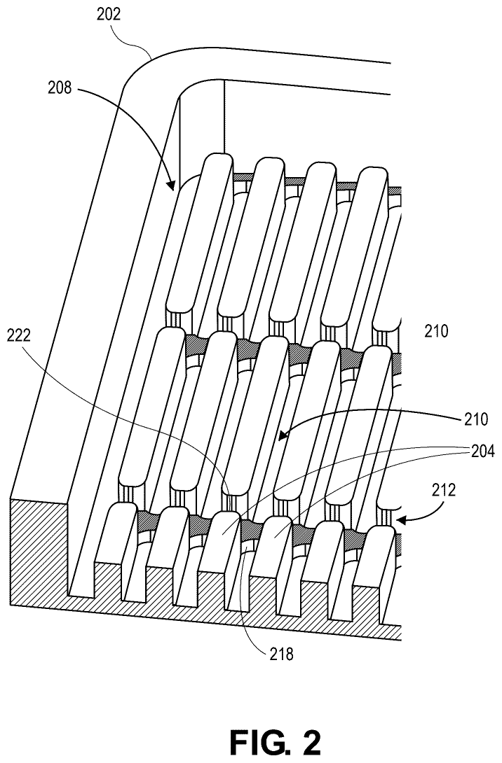

FIG. 2 illustrates cultureware 202 with basin base 208 and fins 204. Rows of fins are separated by gaps 212. In gaps 212 are low-rise fences 218, i.e., inter-gap fences.

Not only are ends 222 of fins 204 rounded, the sides of fences 218 are rounded as well. Fences 218 are concave to grooves 210. This can be simpler to manufacture than squared ends, given that molds are sometimes manufactured from tiny, rotating end mills.

The longitudinal ends of the fins can be squared, rounded, tapered, chamfered, or other geometries.

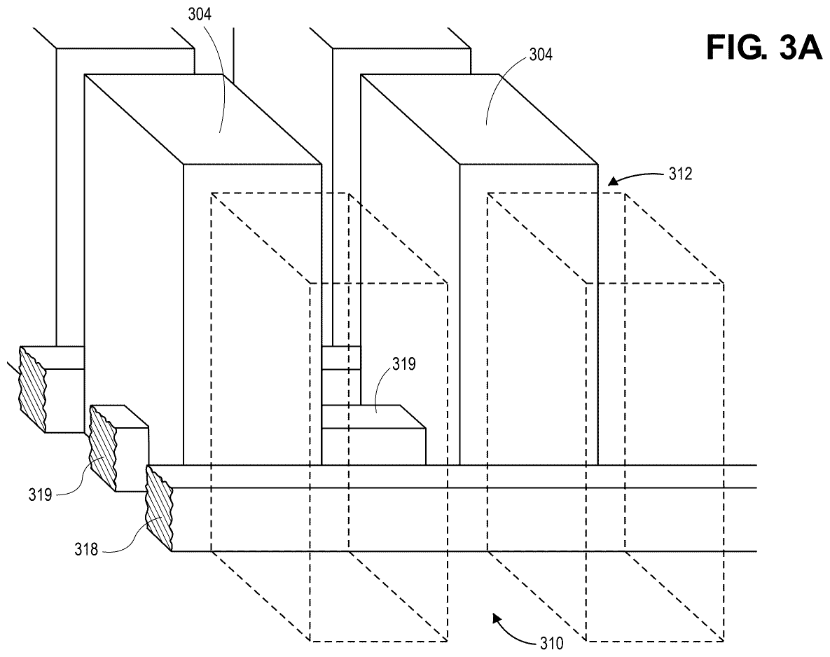

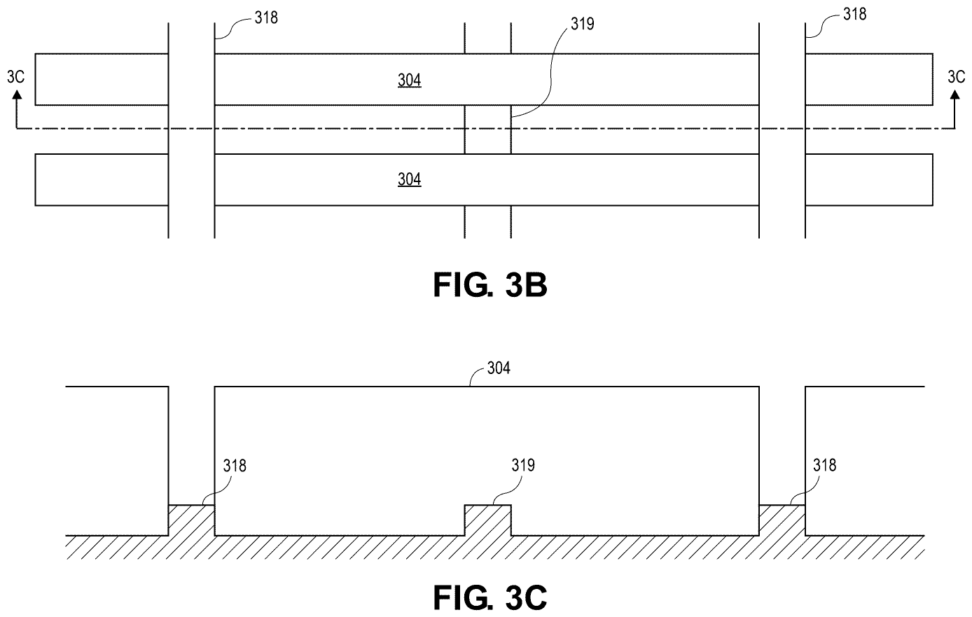

FIGS. 3A-3C illustrate inter-gap and inter-fin low-rise fences. Fins 304 project upward from a basin base, leaving grooves 310 in between. Gaps 312 are between columns of fins 304.

Inter-gap fence 318 projects upward from the basin base and extends continuously through gap 312, from row to row. Fence 318 maintains a rectangular cross section and flat top. It is about ⅕ the height of fins 304.

Inter-fin fences 319 project upward from the basin base and extend between fins 304. Fences 319 are shown at the midpoint of each fin but could be offset to one end. Like fences 318, fences 319 have a rectangular cross section and flat top, and they are the same height as inter-gap fence 318.

In some embodiments, inter-fin fences can be a different cross-section shape or a different height from the inter-gap fences. For example, they may be shorter than inter-gap fences so as to better preserve liquid flow through a groove but also give bubbles a place to dislodge from a bottom of the groove. Meanwhile, the taller inter-gap fences can give bubbles more surface area to work their way up the height of a fin so that they may break free and dissipate.

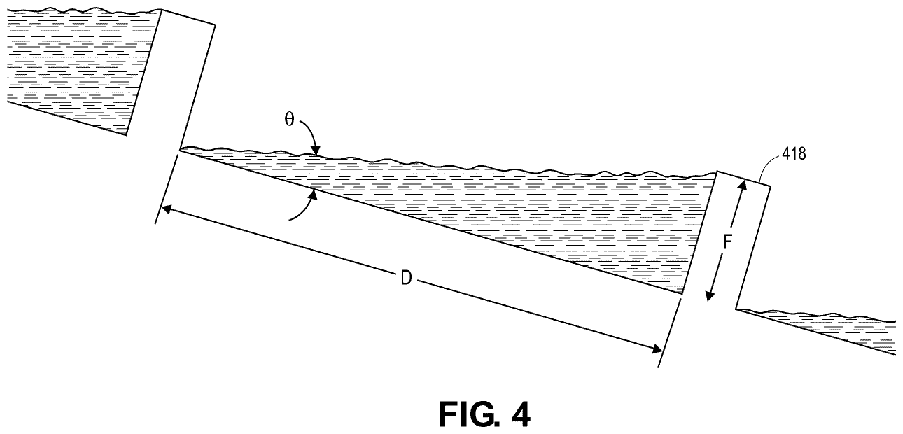

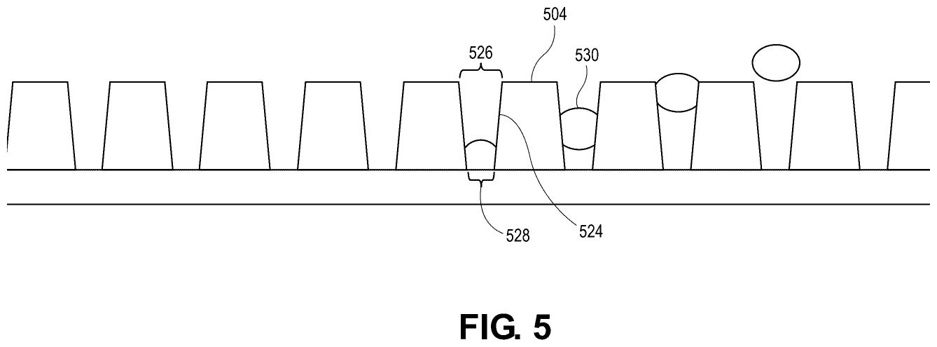

FIG. 4 illustrates a cross-section of multiple low-rise fences at an angle with respect to gravity. This can occur when a cultureware plate is moved. The height, F, of fence 418 is selected so that it can contain liquid without spilling when the cultureware is tilted to angle theta (θ). The height can be calculated by multiplying the tangent of θ by the distance between neighboring fences. For the case of 5°, the height can be is equal-to-or-greater-than tan(5°) (i.e., 0.0875) multiplied by the distance. For example, for an inter-fence distance of 3.0 mm, the height of the fences should be at least 263 μm.

FIG. 5 is a cross-section of fins 504 that taper from their bottoms to their tops, forming wedge like grooves. Bubble 530 is represented as exiting a top of the grooves. Fins 504 have tapered sides 524 such that the grooves between them have narrow bottoms 528 and wide openings 526 at the top.

The fins are shown as tapered at 5° from vertical, but other slope angles, such as 1°, 2°, 3°, 4°, 6°, 7°, 8°, 9°, 10°, 12.5°, 15°, 20°, 25°, 30°, 35°, 40°, or 45°, can work.

Fences may also employ tapering, albeit at a smaller scale. Their angles can be similar to those of the tapered fins.

A technical advantage of tapered fins is that air bubbles may find it energetically favorable to eject themselves from a groove or gap given a taper. Also, a taper may allow an injection molded workpiece to be more easily ejected from a mold.

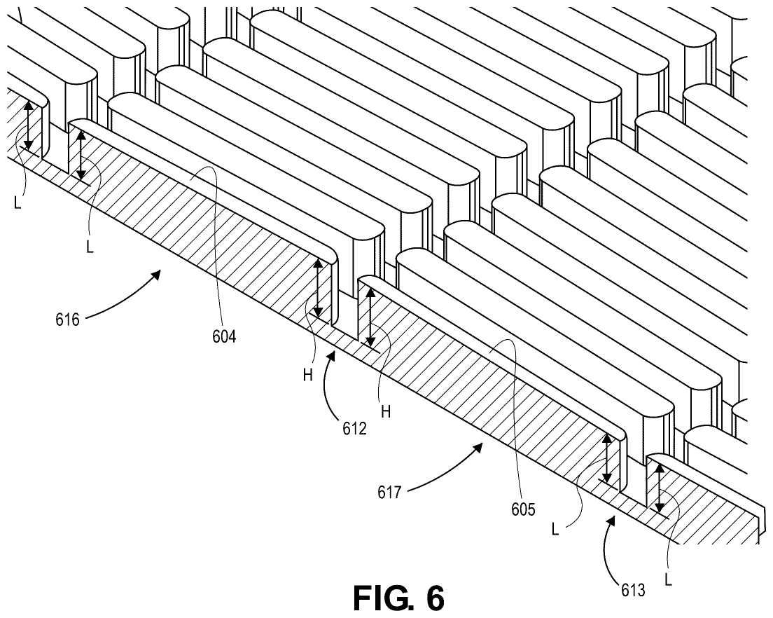

FIG. 6 illustrates fins 604 and 605 in the bottom of cultureware that have tops that slope along their longitudinal lengths. A height H of each fin at gap 612 is taller than a height L of the same fin at gap 613.

Column 616 has fins that all slope in the same direction as fin 604. That is, they slope up when moving from left to right on the page. Meanwhile, column 617 has fins that all slope in the same direction as fin 605, sloping down when moving from left to right on the page. Alternating columns, including columns 616 and 617, have fins with opposite slopes such that a high “H” side of a column is adjacent a high “H” side of an adjacent column, and a low “L” side of a column is adjacent a low “L” side of an adjacent column. This vertical chevron pattern repeats at each column.

A technical advantage of fin heights that slope along their longitudinal lengths is that air bubbles tend to find their way to the highest end of the fins—then merge. When merged, the larger air bubbles are less stable than their constituent bubbles and are more likely to break free to the upper surface of the liquid.

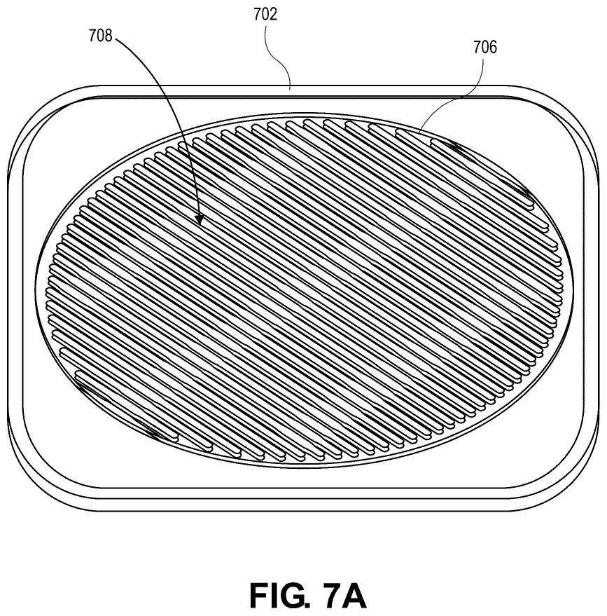

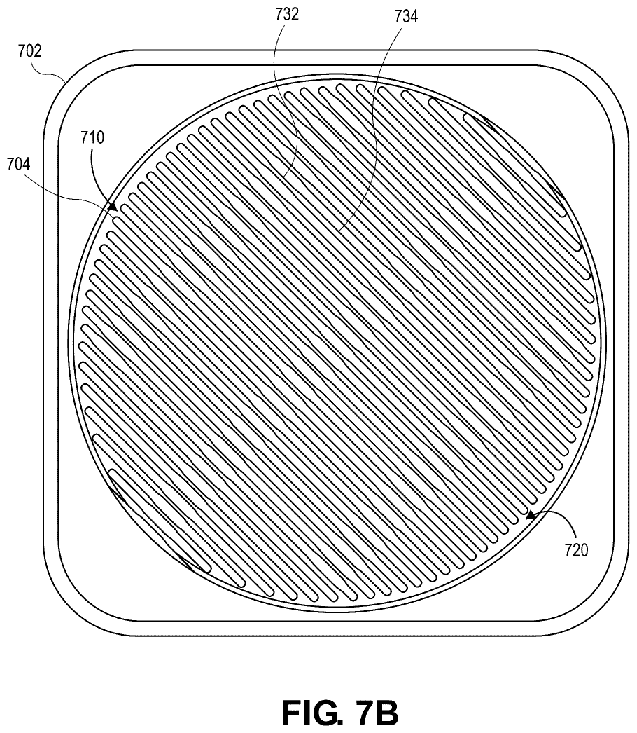

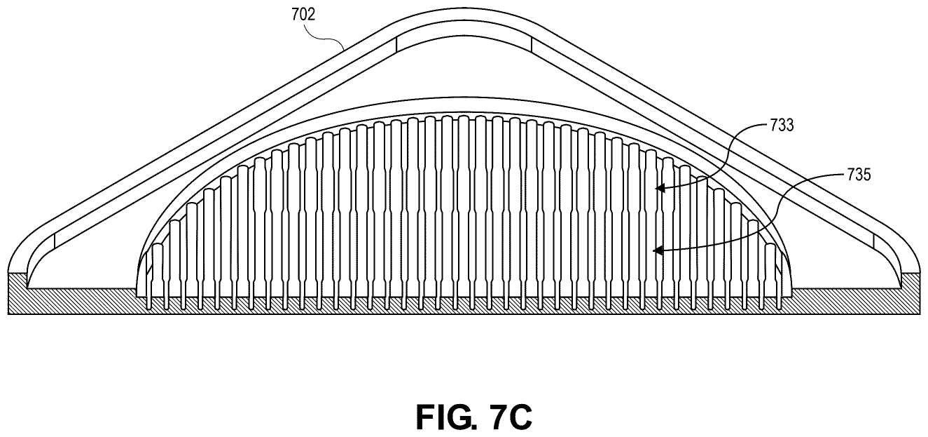

FIGS. 7A-7C illustrate round petri dish 702 cultureware with fins and grooves whose widths vary along their lengths. Petri dish 702 includes sidewall 706 and basin bottom 708 with fins and grooves.

FIG. 7B shows fins 704 projecting up from the basin bottom and forming grooves 710 between them. A lateral (y-axis) width of each fin varies along its length. Minimum fin width 732 is at the fin's narrowest point, and maximum fin width 734 is at the fin's widest point. The minimum and maximum widths of the fins are aligned at the same longitudinal locations.

Peripheral groove 720 surrounds all of the fins at the sidewall perimeter, as fins 704 stop short of sidewall 706 (FIG. 7A). The peripheral groove's depth can be more than, equal to, or less than the depth of the grooves that are between the fins.

FIG. 7C shows that the minimum fin widths widen the adjacent grooves at point 733, and the maximum fin widths, when aligned longitudinally, form pinch point 735 between the neighboring fins.

A technical advantage of pinch points in grooves is that bubbles tend to eject from pinch points, migrating to wider points. At wider points, air bubbles are more likely to break free and disperse. Outer pinch points also eject bubbles toward the peripheral groove, where they have more room to combine with other bubbles and rise to the surface.

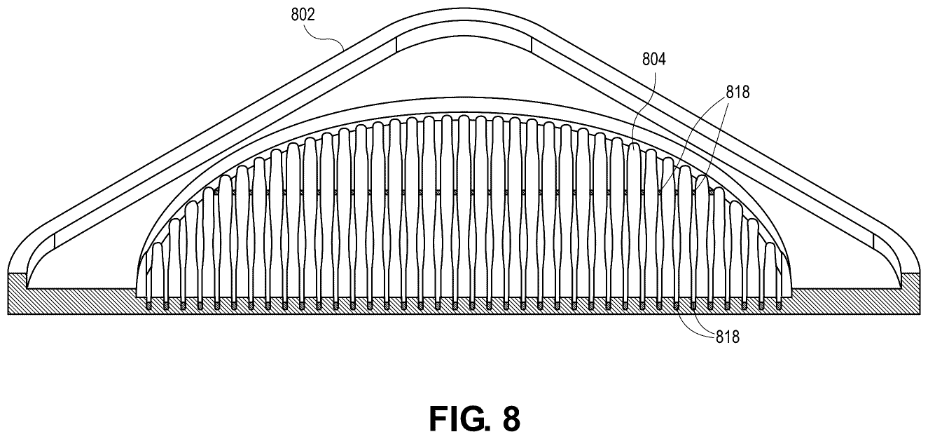

FIG. 8 illustrates portion of well 802 with fins 804 protruding from its basin base. At the fins'widest points where they align, a pinch point is created. Within the pinch points are low-rise fences 818.

The low-rise fences help eject bubbles from the pinch points. They can be combined with longitudinal gaps, tapered fins, rounded fin ends, and other features as discussed to purge bubbles from the cultureware.

In some embodiments, the low-rise fences can be laid at the widest groove points. This may trip up air pockets or keep them from forming in the larger regions.

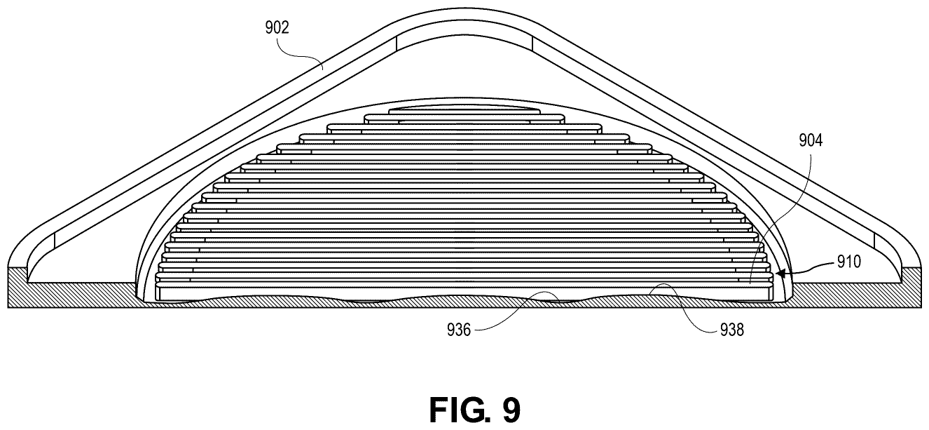

FIG. 9 illustrates portion of well 902 with fins 904 protruding from its basin base, forming grooves 910. The cross section splits a groove longitudinally in order to see the floor of the groove.

Groove bottom 936 undulates up and down, forming peaks 938 and valleys. Peaks 938 among the different grooves align laterally, such that all of the peaks are at the same longitudinal position in the basin bottom.

Bubbles can work their way from the valleys in the grooves to the peaks and be dissipated. In some embodiments, the peaks can go up halfway up the side of the fins. In others, the peaks can rise much lower, for example ⅕ of the height of the fins.

Undulating groove bottoms, longitudinal fin gaps, tapered fins, rounded fin ends, and other features can be combined in an embodiment in order to purge bubbles from closely spaced fins or other elongated protrusions in the bottom of cultureware.

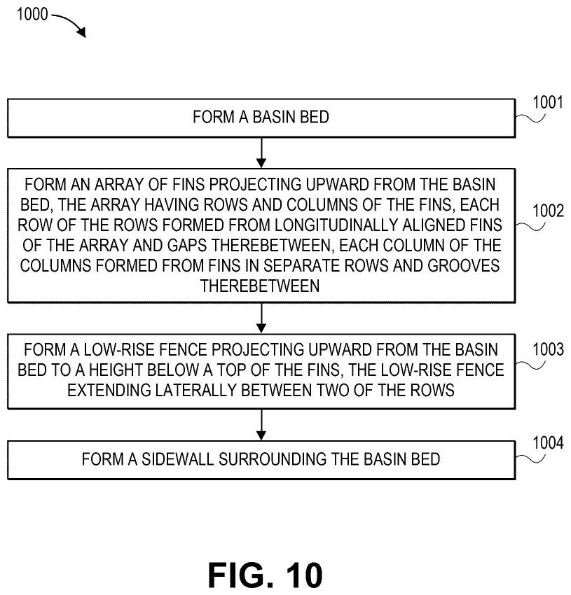

FIG. 10 is a flowchart illustrating process 1000 in accordance with an embodiment. In operation 1001, a basin bed for cultureware is formed. In operation 1002, an array of fins projecting upward from the basin bed is formed, the array having rows and columns of the fins, each row of the rows formed from longitudinally aligned fins of the array and gaps therebetween, each column of the columns formed from fins in separate rows and grooves therebetween. In operation 1003, a low-rise fence projecting upward from the basin bed is formed to a height below a top of the fins, the low-rise fence extending laterally between two of the rows. In operation 1004, a sidewall surrounding the basin bed is formed.

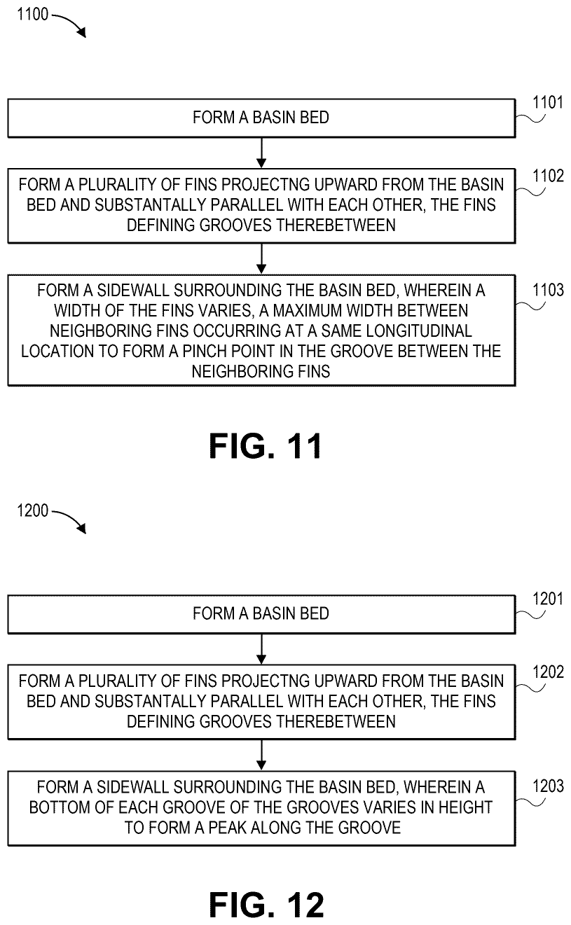

FIG. 11 is a flowchart illustrating process 1100 in accordance with an embodiment. In operation 1101, a basin bed for cultureware is formed. In operation 1102, a plurality of fins projecting upward from the basin bed and substantially parallel with each other is formed, the fins defining grooves therebetween. In operation 1103, a sidewall surrounding the basin bed is formed, wherein a width of the fins varies along its length, a maximum width between neighboring fins occurring at a same longitudinal location to form a pinch point in the groove between the neighboring fins.

FIG. 12 is a flowchart illustrating process 1200 in accordance with an embodiment. In operation 1201, a basin bed for cultureware is formed. In operation 1202, a plurality of fins projecting upward from the basin bed and substantially parallel with each other is formed, the fins defining grooves therebetween. In operation 1203, a sidewall surrounding the basin bed is formed, wherein a bottom of each groove of the grooves varies in height to form a peak along the groove.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” in reference to a temperature or other engineering units includes measurements or settings that are within ±1%, ±2%, ±5%, ±10%, or other tolerances of the specified engineering units as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A cultureware vessel apparatus comprising:
a basin bed;
an array of fins projecting upward from the basin bed, the array having rows and columns of the fins, each row of the rows formed from longitudinally aligned fins of the array and gaps therebetween, each column of the columns formed from fins in separate rows and grooves therebetween;
a low-rise fence projecting upward from the basin bed to a height below a top of the fins, the low-rise fence extending laterally between two of the rows; and
a sidewall surrounding the basin bed.

2. The apparatus of claim 1 wherein the low-rise fence extends between two of the gaps.

3. The apparatus of claim 1 wherein the low-rise fence extends between a pair of the fins.

4. The apparatus of claim 3 wherein the low-rise fence is a first low-rise fence, the apparatus further comprising:
a second low-rise fence extending between the pair of fins.

5. The apparatus of claim 4 wherein a height of the first and second low-rise fences is at least tan(5°) multiplied by a distance between the first and second row-rise fences.

6. The apparatus of claim 1 wherein each fin of the fins has a top that slopes along a longitudinal length of the fin.

7. The apparatus of claim 6 wherein a column of the columns has tops of fins that slope in a same direction.

8. The apparatus of claim 7 wherein alternating columns of the fins have opposite slopes such that a high side of a first column of fins is adjacent a high side of an adjacent column of fins, and a low side of the first column of fins is adjacent a low side of an opposite adjacent column of fins.

9. The apparatus of claim 1 wherein a width of each fin varies, a maximum width between neighboring fins occurring at a same longitudinal location to form a pinch point in the groove therebetween.

10. The apparatus of claim 9 wherein pinch points in multiple of the grooves align laterally.

11. The apparatus of claim 1 wherein a bottom of each groove of the grooves varies in height to form a peak along the groove.

12. The apparatus of claim 11 wherein the peaks along the grooves align laterally.

13. The apparatus of claim 1 wherein a height of the low-rise fence is less-than-or-equal-to half of a height of the fins.

14. The apparatus of claim 1 wherein at least one of the fins has a height between 400 μm and 1000 μm, a width between 300 μm and 500 μm, and a pitch between 200μm and 500 μm.

15. (canceled)

16. The apparatus of claim 1 wherein longitudinal ends of the fins are rounded, tapered, or chamfered.

17. The apparatus of claim 1 wherein the basin bed, the fins, and the fence are formed from a gas-permeable resilient polymer.

18. The apparatus of claim 1 wherein a width of each fin tapers from a bottom to a top of the fin.

19. The apparatus of claim 1 wherein a peripheral groove adjacent the sidewall surrounds all of the fins.

20-21. (canceled)

22. A cultureware vessel apparatus comprising:
a basin bed;
a plurality of fins projecting upward from the basin bed and substantially parallel with each other, the fins defining grooves therebetween; and
a sidewall surrounding the basin bed,
wherein a width of the fins varies, a maximum width between neighboring fins occurring at a same longitudinal location to form a pinch point in the groove between the neighboring fins.

23-24. (canceled)

25. A cultureware vessel apparatus comprising:
a basin bed;
a plurality of fins projecting upward from the basin bed and substantially parallel with each other, the fins defining grooves therebetween; and
a sidewall surrounding the basin bed,
wherein a bottom of each groove of the grooves varies in height to form a peak along the groove.

26-27. (canceled)
